(19) United States
(12) Patent Application Publication
(10) Pub. No.: US 2024/0418714 A1
(43) Pub. Date: Dec. 19, 2024

(54) COMPOSITIONS AND METHODS FOR FORECASTING RESPONSE TO LUPUS NEPHRITIS (LN)THERAPY

(71) Applicant: Children's Hospital Medical Center, Cincinnati, OH (US)

(72) Inventors: Hermine I. Brunner, Cincinnati, OH (US); Michael R. Bennett, Independence, KY (US); Prasad Devarajan, Cincinnati, OH (US)

(21) Appl. No.: 18/602,128

(22) Filed: Mar. 12, 2024

Related U.S. Application Data
(63) Continuation of application No. 15/951,420, filed on Apr. 12, 2018, now abandoned.

(51) Int. Cl. G01N 33/564; A61K 31/664; G16H 10/40

(52) U.S. Cl. CPC G01N 33/564; G16H 10/40; A61K 31/664; G01N 2800/104; G01N 2800/50; G01N 2800/52; G01N 2800/54

(57) ABSTRACT

Disclosed herein are methods for forecasting response to lupus nephritis (LN) therapy in individual diagnosed with childhood-onset SLE (cSLE). The methods may include the step of detecting each protein in a protein set in a sample obtained from an individual in need thereof. The protein set may include ceruloplasmin, kidney injury molecule 1 (KIM-1), monocyte chemotactic protein 1 (MCP-1), adiponectin, hemopexin, and NGAL.

### STATEMENT REGARDING FEDERALLY-SPONSORED RESEARCH

This invention was made with government support under AR065098, DK096418 and AR059509 awarded by the National Institutes of Health. The government has certain rights in the invention.

### BACKGROUND

Systemic Lupus Erythematosus (SLE) is a multi-system inflammatory autoimmune disease, and lupus nephritis (LN) is one of the main determinants of poor prognosis (1-4). Although data from large-scale epidemiological studies are lacking, an estimated 10% of the children and adolescents will develop end-stage renal disease (ESRD) within 10 years of LN diagnosis (5), and 22% of children in ESRD from LN will die within 5 years of requiring renal replacement therapy (6). A major factor leading to such dissatisfactory LN outcomes is a lack of non-invasive clinical and laboratory measures to accurately gauge LN status in terms of activity and response to therapy.

### BRIEF SUMMARY

Disclosed herein are methods for forecasting response to lupus nephritis (LN) therapy in individual diagnosed with childhood-onset SLE (cSLE). The methods may include the step of detecting each protein in a protein set in a sample obtained from an individual in need thereof. The protein set may include ceruloplasmin, kidney injury molecule 1 (KIM-1), monocyte chemotactic protein 1 (MCP-1), adiponectin, hemopexin, and NGAL

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Squares represent means of biomarkers in the group of non-responders (n=50). Dots represent means among responders to therapy. Panel (A) Neutrophil gelatinase associated lipocalin, NGAL; Panel (B) ceruloplasmin, CP; Panel (C) kidney injury molecule 1, KIM-1; Panel (D) monocyte chemotactic protein, 1 MCP-1; Panel (E) adiponectin, ADIPO; Panel (F) hemopexin, HPX; Panel (G) transferrin, TF; Panel (H) alpha-1-acid glycoprotein AGP; Panel (I) lipocalin-like prostaglandin D synthase, LPGDS. A “*” and “**” indicates the difference of means between responders and non-responders is statistically significant with its p-value<0.05 and 0.01, respectively.

FIG. 2. Patterns of differences in the urine biomarker levels over time between responders and non-responders to LN therapy. P-values from mixed model analysis are compared between groups. Neutrophil gelatinase associated lipocalin, NGAL; kidney injury molecule 1, KIM-1; monocyte chemotactic protein, 1 MCP-1; alpha-1-acid glycoprotein AGP; transforming growth factor beta, TGF-β; Fatty acid-binding protein, LFABP; hepcidin; vitamin D binding protein, VDBP; lipocalin-like prostaglandin D synthase, LPGDS. Clear=P>0.01; light gray=0.05<P<0.1; medium gray=0.005<P<0.05; dark gray=P<0.005.

FIG. 3. Changes of urine biomarkers (log transformed) means from baseline to month 3 in patients with response to LN therapy and changes of urine biomarkers (log transformed) means from baseline to month 3 in patients with non-response. A indicates the difference of means between respondents and non-respondents is statistically significant with its p-value<0.01. Panel (B): Area under the receiver operating characteristic curve (AUC) between responders and non-responders over time. The RAIL biomarkers biomarkers (NGAL, MCP-1, adiponectin, Kim-1, ceruloplasmin, hempexin) combined were excellent to discriminate responders from non-responders when considering all time points. However, adiponectin, AGP, transferrin and adiponectin and VDBP individually had excellent ability to anticipate treatment response as early as month 3. Only for biomarkers or combination of biomarkers with outstanding accuracy (AUC>0.9) values are shown. Alpha-1-acid glycoprotein AGP; kidney injury molecule 1, KIM-1; lipocalin-like prostaglandin D synthase, LPGDS. monocyte chemotactic protein, 1 MCP1; neutrophil gelatinase associated lipocalin, NGAL; transforming growth factor β, TGF-β; vitamin binding protein, VDBP.

FIG. 4. Changes of the biomarkers from baseline to a follow-up month with CTX treatment and changes of the biomarkers from baseline to a follow-up month with MMF treatment. Presentation of a “*” and “**” indicates that the difference of means between responders and non-responders is statistically significant with its p-value<0.05 and 0.01 respectively. Neutrophil gelatinase associated lipocalin, NGAL; ceruloplasmin, CP; kidney injury molecule 1, KIM-1; monocyte chemotactic protein, 1 MCP1; Adiponectin, ADIPO; hemopexin, HPX.

FIG. 5. Mean biomarker levels by response over time. Values are ln transformed and not corrected by urine creatine.

FIG. 6. Assessment of moderations (or interactions) of race and gender in affecting associations of urine biomarkers vs. response.

FIG. 7. Spearman correlations for non-adjusted urine biomarker levels with extra-renal disease activity as measured by the SLEDAI

FIG. 8. Assessment of moderations (or interactions) of using ACE/ARB in affecting the associations of urine biomarkers vs. response.

FIG. 9. Changes of non-adjusted biomarkers (follow up vs. baseline) with CTX or MMF therapy. Osteopontin is omitted due to non-responsiveness with LN course.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

Several urine biomarkers have been described that hold much promise to improve the surveillance of LN compared to the current clinical and laboratory measures (7, 8). Applicant have shown that the urine concentrations of adiponectin, ceruloplasmin, hemopexin, kidney injury molecule 1 (KIM-1), monocyte chemotactic protein 1 (MCP-1), and neutrophil gelatinase associated lipocalin (NGAL) can be used to calculate the Renal Activity Index in Lupus (RAIL); this index has excellent accuracy in estimating histological activity of LN in both children and adults (9, 10). Although past research suggests that the RAIL-biomarkers and some of the other biomarkers mentioned above are suited to capture or even anticipate LN response to therapy, this has not been studied well in children. Further, the importance of patient demographics, LN histology and medication usage on the responsiveness of these urine biomarkers to capture clinically relevant improvement of LN has not been studied in depth.

Disclosed are non-invasive methods for forecasting response to lupus nephritis (LN) therapy in individual diagnosed with childhood-onset SLE (cSLE). The method may comprise the step of detecting each protein in a protein set in a sample obtained from the individual, wherein the protein set may comprise ceruloplasmin, kidney injury molecule 1 (KIM-1), monocyte chemotactic protein 1 (MCP-1), adiponectin, and hemopexin.

In one aspect, the method of claim may comprise the steps of calculating a Renal Activity Index for Lupus (RAIL) score based on the expression level of each protein in the protein set; and classifying said individual as an LN therapy non-responder or an LN therapy responder, wherein said expression level is determined in a urine sample obtained from said individual, wherein said sample is obtained at least about three months or at least about six months after a treatment for LN is initiated; wherein if said individual is an LN therapy non-responder, said individual is treated with an alternative therapy or subjected to a repeat biopsy, and if said individual is an LN therapy responder, said treatment is maintained.

In one aspect, the method may comprise the step of comparing the expression level of each protein in said protein step to a control value, wherein an increase in said levels indicates that said individual is likely to be a non-responder, and wherein a decrease in the levels of said protein set compared to said control value indicates that said individual is likely to be a responder.

In one aspect, the protein set may further comprise neutrophil gelatinase-associated lipocalin (NGAL).

In one aspect, the sample may be obtained at least six months post-treatment initiation. In another aspect, the sample may be obtained at least six months post-treatment initiation.

In one aspect, the treatment may be mycophenolate mofetil (MMF), cyclophosphamide (CTX), or a combination thereof. In other aspects, the treatment may further comprise an angiotensin system-blocking drug.

In one aspect, the LN may be proliferative LN.

In one aspect, the RAIL score may be calculated from the log-transformed and urine creatine standardized concentrations as follows: P-RAIL=−4.29*NGAL-0.06*ceruloplasmin+0.89*MCP-1+0.18*adiponectin−0.65*hemopexin+0.62*KIM-1, wherein a higher score indicates that said individual is likely to be a non-responder, and wherein a lower score indicates that said individual is likely to be a responder. Such calculation may be calculated using a computer.

In one aspect, the individual may be less than 18 years of age.

In one aspect, the method may further comprise the step of contacting said sample with a composition comprising a plurality of detection agents specific for ceruloplasmin, kidney injury molecule 1 (KIM-1), monocyte chemotactic protein 1 (MCP-1), adiponectin, and hemopexin. In certain aspects, the detection agent may be an antibody. One of ordinary skill in the art will readily appreciate antibodies suitable for use with the disclosed methods.

In one aspect, a kit for forecasting response to lupus nephritis (LN) therapy in individual diagnosed with childhood-onset SLE (cSLE) is disclosed. The kit may comprise a set of detection agents consisting of detection agents capable of detecting the expression products of ceruloplasmin, kidney injury molecule 1 (KIM-1), monocyte chemotactic protein 1 (MCP-1), adiponectin, and hemopexin, and optionally, NGAL. The kit may further comprise a computer product for calculating a RAIL score, wherein said RAIL score is predictive of LN response therapy.

In one aspect, a composition comprising a plurality of detection agents specific for a protein set comprising ceruloplasmin, kidney injury molecule 1 (KIM-1), monocyte chemotactic protein 1 (MCP-1), adiponectin, and hemopexin, and optionally NGAL, is disclosed. The detection agents may be antibodies. In one aspect, the detection agents may be provided in a form selected from a solution-based composition, such as a solution containing a plurality of antibodies, or, in other aspects, a substrate-based composition such as a plurality of antibodies affixed to a substrate such as plastic, paper, glass, or the like, which may be configured for contact with a biological sample obtained from an individual which may be further used with the disclosed methods for determining response to LN treatment.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Methods: Starting from the time of kidney biopsy, patients with childhood-onset SLE who were diagnosed with LN were studied serially. Levels of 15 biomarker were measured in random spot urine samples, including adiponectin, alpha-1-acid glycoprotein (AGP), ceruloplasmin, hemopexin, hepcidin, kidney injury molecule-1 (KIM-1), monocyte chemotactic protein-1 (MCP-1), lipocalin-like prostaglandin synthase (LPGDS), transforming growth factor beta (TGF-β), transferrin, and vitamin-D binding protein (VDBP).

Results: Among 87 patients [mean age 15.8 years] with LN, there were 37 treatment-responders and 50 non-responders based on the American College of Rheumatology criteria. At the time of kidney biopsy, levels of TGF-β (p<0.0001) and ceruloplasmin (P=0.006) were had significantly lower levels among responders than non-responders; less pronounced differences were present for AGP, hepcidin, LPGDS, transferrin and VDBP (all P<0.05). By month 3, responders experienced marked decreases of adiponectin, AGP, transferrin and VDBP (all P<0.01) and mean levels of these biomarkers were all outstanding (area under the ROC curve>0.9) for discriminating responders from non-responders. Patient demographics and extra-renal disease did not influence differences in biomarker levels between response groups.

Conclusion: Low urine levels of TGF-β and ceruloplasmin at baseline and marked reduction of AGP, LPGDS, transferrin or VDBP and combinations of other select biomarkers by month 3 are outstanding predictors for achieving remission of LN. If confirmed, these results can be used to personalize LN therapy.

Several urine biomarkers have been described that hold much promise to improve the surveillance of LN compared to the current clinical and laboratory measures (7, 8). Applicant has shown that the urine concentrations of adiponectin, ceruloplasmin, hemopexin, kidney injury molecule 1 (KIM-1), monocyte chemotactic protein 1 (MCP-1), and neutrophil gelatinase associated lipocalin (NGAL) can be used to calculate the Renal Activity Index in Lupus (RAIL); this index has excellent accuracy in estimating histological activity of LN in both children and adults (9, 10). Other promising biomarkers of LN include alpha-1-acid glycoprotein (AGP), cystatin-C, hepcidin, lipocalin-like prostaglandin D synthase (LPGDS), liver type fatty acid-binding protein 1 (LFABP), osteopontin, transforming growth factor beta (TGF-β), transferrin, and vitamin D binding protein (VDBP) (10-16).

Although past research suggests that the RAIL-biomarkers and some of the other biomarkers mentioned above are suited to capture or even anticipate LN response to therapy, this has not been studied well in children. Further, the importance of patient demographics, LN histology and medication usage on the responsiveness of these urine biomarkers to capture clinically relevant improvement of LN has not been studied in depth. Applicant hypothesized that a selection of urine biomarkers can be used to anticipate the course of LN in children accurately, with dependency on the histological severity of LN but irrespective of background medication use, patient demographics or extra-renal disease activity.

### Materials & Methods

### Patients

Patients diagnosed with childhood-onset SLE (cSLE) (17) who required a kidney biopsy as part of standard of care participated in this longitudinal study. Random spot urine samples were collected at the time of kidney biopsy and in regular intervals thereafter for up to 24 months. Prospectively, relevant clinical information and traditional measures of LN were recorded, including the glomerular filtration rate (GFR) (18, 19) and the protein to creatinine ratio (PC-ratio) in a random urine sample. All patients received therapy for cSLE at the time of the urine collection and biopsy.

The renal domain scores of the Systemic Lupus Disease Activity Index (SLEDAI; range 0-16; 0=inactive LN) (20) and the British Isles Lupus Activity Group (BILAG) index (21) were completed to serve as measures of LN clinical activity. Applicant also measured extra-renal disease activity using the SLEDAI as previously described (22). The study was approved by the institutional review boards of all of the participating institutions; patients and/or caretakers provided informed assent and consent prior to commencement of any study related activities.

### Kidney Histology and Response to Therapy

The histological characteristics of available kidney biopsies were all interpreted in a blinded fashion by an expert nephropathologist (DW) as per the International Society for Nephrology/Renal Pathology Society (ISN/RPS) Classification (23, 24). In line with what has been proposed by the American College of Rheumatology (ACR), complete response to LN therapy was defined as the presence of an inactive urine sediment plus decrease of proteinuria (estimated by PC-ratio) to <0.2 grams per day plus normal or stable GFR based on the modified Schwartz formula (25, 26).

### Urinary Biomarker Assays

The following 15 biomarkers were assayed: adiponectin, AGP, ceruloplasmin, cystatin-C, hemopexin, hepcidin, KIM-1, LPGDS, LFABP, MCP-1, NGAL, osteopontin, TGF-β, transferrin and VDBP. Laboratory personnel assaying the biomarkers were blinded to clinical and histological information. Spun urine samples were stored at 0° C. within 1 hour of collection and frozen at −80° C. prior within 24 hours prior to batch processing.

Unless stated otherwise, biomarkers were quantified using commercial ELISA kits as per the manufacturers' instructions, and a four-parameter logistic curve-fit was used to fit the standard curve. The inter-assay and intra-assay variability of these assays is expressed in percent of the coefficient of variation [CV inter/intra].

Adiponectin [CV inter/intra: 4.0%/9.9%] was measured using the Quantikine ELISA Human HMW Adiponectin/Acrp30) (R&D Systems, Minneapolis, MN); AGP [CV inter/intra: 5.0%/8.5%] by ELISA (R&D Systems, Minneapolis, MN); ceruloplasmin [CV inter/intra: 4.1%/7.1%] by ELISA (Assaypro, St.Charles, MO); hemopexin [CV inter/intra: 4.8%/7.3%] with the Assay Max Human Hemopexin ELISA Kit (Assaypro, St. Charles, MO); and hepcidin-25 [CV inter/intra: 3.5%/3.4%] was measured by ELISA (R&D Systems, Minneapolis, MN). The KIM-1 assay was constructed using commercially available reagents (Duoset DY1750, R & D Systems, Minneapolis, MN) as described previously (27). Applicant quantified LFABP [CV inter/intra: 6.1%/10.9%] by ELISA (CMIC Co., Tokyo, Japan); MCP-1 [CV inter/intra: 5.0%/5.9%] by ELISA (R&D Systems, Minneapolis, MN); NGAL [CV inter/intra: 1.0%/9.1%] was measured by ELISA (Human NGAL ELISA; Bioporto, Grusbakken, Denmark); osteopontin [CV inter/intra: 7.8%/9.0%] with the DuoSet Human EPCR kit (R&D Systems, Minneapolis, MN); and VDBP [CV inter/intra: 5.1%/6.2%] by ELISA (R&D Systems, Minneapolis, MN, respectively.

TGF-β [CV inter/intra: 2.6%/8.3%] was measured by ELISA (R&D Systems, Minneapolis, MN) after acid activation. Briefly, 20 μL of 1N HCl was added to 100 μL of urine sample, mixed by inversion and incubated at room temperature for 10 minutes. Next, the acidified sample was neutralized by adding 20 μL of 1.2 N NaOH/0.5 M HEPES, then the assay was immediately run per manufacturer's instructions [CV inter/intra: 2.0%/7.8%]. Using immunonephelometry (Siemens, BNII, Munich, Germany) Applicant measured cystatin-C [CV inter/intra: 2.5%/2.3%], transferrin [CV inter/intra: 3.4%/2.5%] and LPGDS [CV inter/intra: 2.3%/6.5%]. Applicant also determined levels of urine creatinine using an enzymatic creatinine assay [CV inter/intra: 0.65%/4.48%] on a Dimension RXL Clinical Analyzer (Siemens, Munich, Germany).

Raw concentrations of the urine biomarkers (in ng/ml for NGAL, AGP, CP, LFABP, VDBP, osteopontin, hemopexin, and hepcidin; in pg/ml for adiponectin, KIM-1, MCP-1 and TGF-β; in ng/dL: for transferrin and LPDGS; in ng/L for cystatin-C) are presented as well as biomarker concentrations standardized for urine creatinine levels (in mg/mL).

### Statistical Analysis

All biomarker levels were found right skewed in their distributions but their (nature) log transformed variables were symmetrically distributed and fit the conditions for parametric statistical models. Hence, all the analyses were performed using log transformed biomarker levels and variation of estimates was measured by the standard error (SE) or standard deviations (SD).

The primary statistical model was a mixed effect model. In particular, each dependent variable, i.e. the log transformed biomarker, was assessed for its associations to the major fixed effects of interest, the response effect (yes vs. no), the time effect (a categorical variable of months 0, 3, 6, 9 and 12), and its interaction in the mixed effect model. A random effect was used to account for within person correlation caused by the repeated measurements over the visits. Post-hoc means of the dependent variable were effect model framework. Mixed effect models were repeated after adjusting biomarker levels by urine creatinine concentrations. Since the findings from the analyses considering urine creatinine adjusted biomarkers agreed with those based on unadjusted urine biomarkers, only results from unadjusted biomarkers are presented herein.

The study also included subset analyses using the primary mixed effect models in subgroups stratified by LN Class as well as the treatment with either cyclophosphamide (CTX) or mycophenolate mofetil (MMF). Applicant assessed differences in biomarker levels between baseline and month 3 (22). To test whether the association between the dependent variable and the response effect were importantly influenced by possible moderators (age, gender, race, angiotensinogen blocking medications), Applicant modified the mixed effect models by letting the moderator interact with the response factor, while nesting them under the time effect.

Receiver operating characteristic (ROC) curves were used to assess the performance of individual biomarkers in discriminating responders from non-responders. The overall accuracy of each biomarker was evaluated using the areas under the ROC curve (AUC). It was considered outstanding, excellent, good, and fair if the AUC was in the range of 0.9-1.0, 0.81-0.90, 0.71-0.80, and 0.61-0.70, respectively.

For the RAIL panel, Applicant the multiple logistical regression model to predict response using levels of NGAL, MCP-1, KIM-1, ceruloplasmin, adiponectin and hemopexin as predictors; and then assess its ROC curve and AUC using the logit score (or RAIL score) were calculated from the multiple logistical regression model, as previously described (28). The performance of the RAIL combinatorial biomarkers is presented both adjusted and unadjusted by urine creatinine.

Applicant calculated Spearmen's correlation coefficients to assess relationships between numerical variables, Chi-square tests to compare rates between groups, and sensitivities and specificities to assess diagnostic accuracies of individual biomarkers. In addition, categorical and numerical variables at baseline were summarized using frequency in % and mean (standard deviation or SD) respectively. All statistical analyses were computed using a SAS 9.4 (SAS, Cary, NC) package. P-values<0.05 were considered statistically significant.

### Results

### Patient Characteristics & Features of Kidney Biopsy

Details about the study cohort are provided in Table 1. In brief, 87 patients with LN were included in this study; all required a kidney biopsy as part of clinical care for cSLE (17). The patients' mean [SD] age at the time of kidney biopsy was 15.6 [2.9] years, and the average extra-renal disease activity as measured by the SLEDAI was 6.3 [5.8]. None had ISN/PRS Class 1 or Class 6 LN. MMF and CTX at recommended doses (22) were often used for LN therapy, and the majority of the patients were prescribed an angiotensin system blocking drug soon after their kidney biopsy.

TABLE 1
Demographics and clinical information of the patients at the time of
urine collection and the time of kidney biopsy. Values are arithmetic means (SD), unless
stated otherwise.

| | | | Responders, | Nonresponders, | |
|---|---|---|---|---|---|
| Variable | Category | Total, n = 87 | n = 37 | n = 50 | p## |
| Sex, n (%) | Female | 68 (78.2) | 27 (73.0) | 41 (82.0) | 0.314 |
| Race/ethnicity, n (%) | White | 29 (33.3%) | 19 (51.4) | 10 (20.0) | 0.015 |
| | Black | 33 (38.0) | 12 (32.4) | 21 (42.0) | |
| | Hispanic | 10 (11.5) | 2 (5.4) | 8 (16.0) | |
| | Mixed racial and others | 15 (17.2) | 4 (10.8) | 11 (22.0) | |
| Medications started for | Mycophenolate mofetil | 47 (54.0) | 17 (46.0) | 30 (60.0) | 0.194 |
| LN therapy around | Azathioprine | 7 (8.1) | 5 (13.5) | 2 (4.0) | |
| kidney biopsy, n (%) | Cyclophosphamide | 33 (37.9) | 15 (40.5) | 18 (36.0) | |
| Angiotensin system- | Yes | 51/87 (58.6) | 20 (54) | 31 (62) | 0.202 |
| blocking drug, n (%) | | | | | |
| GFR, ml/min/m² | | 135.6 (57.4) | 141.0 (66.4) | 131.6 (50.1) | 0.459 |
| Renal SLEDAI | | 8.0 (5.2) | 5.4 (4.7) | 9.8 (4.7) | <0.0001 |
| Renal BILAG | | 9.9 (4.0) | 8.2 (5.1) | 11.2 (2.1) | 0.0003 |
| Microalbumin/ | | 1.16 (2.04) | 0.80 (2.23) | 1.54 (1.83) | 0.122 |
| creatinine ratio** | | | | | |
| ISN/RPS, n (%)# | Class 2 | 13 (14.9) | 5 (13.5) | 8 (16.0) | 0.634 |
| | Class 3 or 4 | 47 (54.0) | 22 (59.5) | 25 (50.0) | |
| | Class 5 | 27 (31.0) | 10 (27.0) | 17 (34.0) | |
| NIH—AI^{‡} | | 7.6 (6.5) | 7.7 (6.0) | 7.5 (6.9) | 0.917 |
| NIH—CI^{Δ} | | 1.6 (1.9) | 1.6 (1.4) | 1.6 (2.1) | 0.952 |
| Extrarenal SLEDAI* | | 6.7 (6.8) | 3.8 (3.3) | 8.8 (8.0) | 0.004 |

##p values are from t tests to compare means or chi square tests to compare rates (in %).
#International Society for Nephrology/Renal Pathology Society Class; there were no biopsies consistent with Class 1 or 6.
^{‡}US National Institutes of Health (NIH) Activity Index; range 0-24; 0 = inactive LN; available in only 76 patients.
^{Δ}NIH Chronicity Index; range 0-12; 0 = LN without chronic changes; available in only 62 patients.
*Measured by the SLEDAI summary score minus the SLEDAI renal domain score.
**Natural log transformed.
GFR: glomerular filtration rate;
LN: lupus nephritis;
SLEDAI: System Lupus Disease Activity Index;
BILAG: British Isles Lupos Activity Group.

### Responders Versus Non-Responders to LN Therapy

Of the 87 patients enrolled, 37 patients showed response and 50 patients failed to respond to treatment. At baseline, responders did not differ significantly in terms of histological activity (p=0.917) and histological chronicity (p=0.952) as measured by the NIH Activity (NIH-AI) and Chronicity Indices (NIH-CI) (29), respectively. Likewise, there were no important baseline differences between groups for renal function (GFR; P=0.459) or the degree of proteinuria (PC-ratio; p=0.255).

As early as month 3 of LN therapy, there were significant differences between responders and non-responders in the change of the PC-ratio from baseline (mean+SE; responders vs. non-responders: −1.17±1.24 vs. 1.08±1.72; P=0.002), and differences in proteinuria persisted over time. Of note, non-responders had markedly higher extra-renal disease activity compared to the responder group at baseline (Table 1).

### Select Urine Biomarkers Differ Between Responders and Non-Responders at the Time of Kidney Biopsy

At the time of kidney biopsy, mean concentrations of seven of the included biomarkers significantly differed with response status (non-responders, responders) (Table 2). The most pronounced differences were observed for TGF-β and ceruloplasmin (both P<0.006), followed by transferrin, AGP, VDBP, hepcidin, and LPGDS. MCP-1 and NGAL showed only trends towards higher levels among non-responders at baseline. Notably, with the exception of hepcidin, urine concentrations of all biomarkers were higher among non-responders than responders.

TABLE 2
Biomarker levels differences at the time of biopsy among 37
responders and 50 nonresponders. Values are geometric means ± SE.

| | Biomarker Levels at the Time of Biopsy | | |
|---|---|---|---|
| Biomarker | Nonresponders | Responders | p* |
| TGF-β | 3.74 ± 0.21 | 2.61 ± 0.22 | <0.0001 |
| Ceruloplasmin | 9.76 ± 0.29 | 8.58 ± 0.30 | 0.006 |
| Transferrin | 2.12 ± 0.32 | 0.93 ± 0.34 | 0.012 |
| AGP | 11.44 ± 0.36 | 10.24 ± 0.38 | 0.023 |
| VDBP | 6.65 ± 0.31 | 5.65 ± 0.32 | 0.027 |
| Hepcidin | 6.88 ± 0.60 | 8.70 ± 0.62 | 0.037 |
| LPGDS | 6.19 ± 0.31 | 5.30 ± 0.28 | 0.044 |
| MCP-1 | 6.87 ± 0.21 | 6.28 ± 0.22 | 0.057 |
| NGAL | 3.77 ± 0.22 | 3.20 ± 0.23 | 0.079 |
| KIM-1 | 7.37 ± 0.22 | 7.10 ± 0.23 | 0.402 |
| Osteoponin | 4.66 ± 0.32 | 4.30 ± 0.34 | 0.432 |
| Hemopexin | 8.10 ± 0.25 | 7.82 ± 0.26 | 0.443 |
| Cystatin-C | 4.35 ± 0.20 | 4.10 ± 0.26 | 0.451 |
| Adiponectin | 10.65 ± 0.16 | 10.27 ± 0.37 | 0.463 |
| LFABP | 3.10 ± 0.26 | 3.09 ± 0.31 | 0.975 |

*p values are computed using mixed effect models.
NGAL: neutrophil gelatinase associated lipocalin;
KIM-1; kidney injury molecule 1;
MCP-1: monocyte chemotactic protein 1;
AGP: α-1-acid glycoprotein;
TGF-β: trans-forming growth factor-β;
LFABP: liver-type fatty acid-binding protein 1;
VDBP: vitamin D binding protein;
LPGDS: lipocalin-like prostaglandin D synthase.

### Mean Urine Biomarker Concentrations Differ Over Time by Responder Status

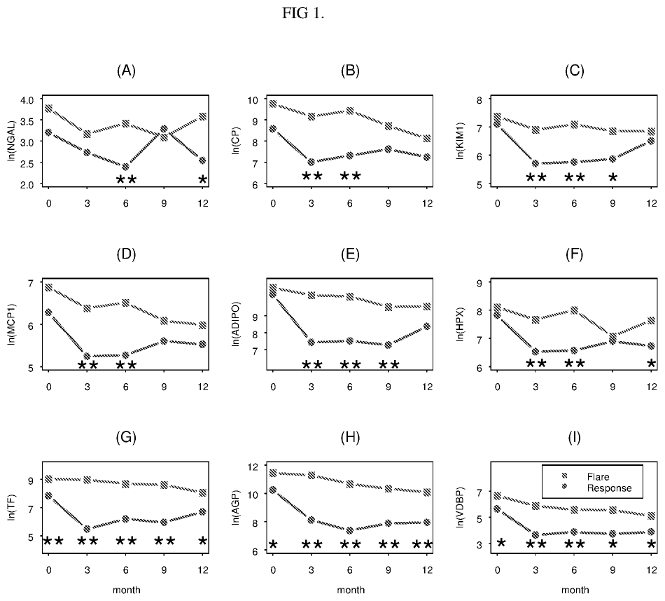

FIG. 1 depicts mean concentrations of the six biomarkers included in the RAIL (Panels A-F) and of three other biomarkers (transferrin, AGP, VDBP in Panels G-I) that markedly differed with response status. All RAIL biomarkers, except for NGAL, differed significantly by responder status at month 3. NGAL showed only significant differences at month 6.

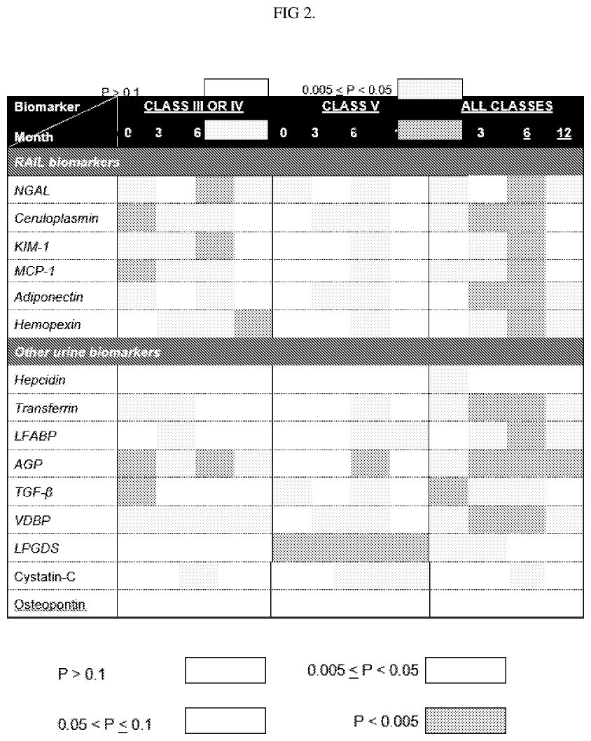

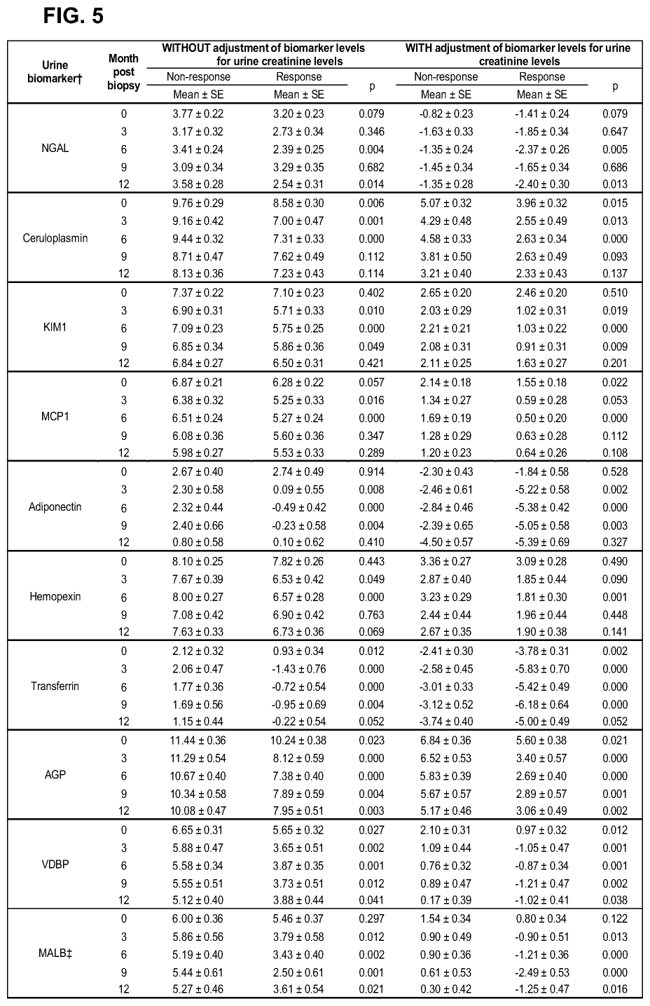

FIG. 2 summarizes differences in biomarker levels between response groups. Although levels of TGF-β and, to a lesser degree, hepcidin as well as LFABP all significantly differed between responders and non-responders at the time of biopsy, only TGF-β and LFABP continued to show significant differences between response groups at month 3 and 6. Osteopontin did not significantly differ between response groups at any of the time points (for details see FIG. 5).

### Differences In Urinary Biomarker Levels Under Consideration of LN Severity

Biomarker patterns over time between responders and non-responders showed dependency on LN severity as defined by the ISN/PRS class. Given the limited numbers of patients with Class 2 LN, FIG. 2 only shows the results of these analyses for proliferative LN (Class 3 or 4) and pure membranous LN (Class 5).

By month 3, the RAIL biomarkers levels differed with responder-status, particularly in proliferative LN. There were also significant differences in the urine levels (p<0.05) of AGP and transferrin for more than one time point between responders and non-responders with proliferative LN.

For pure Class 5 LN, LPGDS levels differed most markedly with response status (all p<0.0001) but VDBP and adiponectin levels also significantly different for more than one time point between responders and non-responders. Additional details are provided in FIG. 5.

### Absolute Changes in Biomarker Levels Between Month 0 and Month 3 by Responder Status

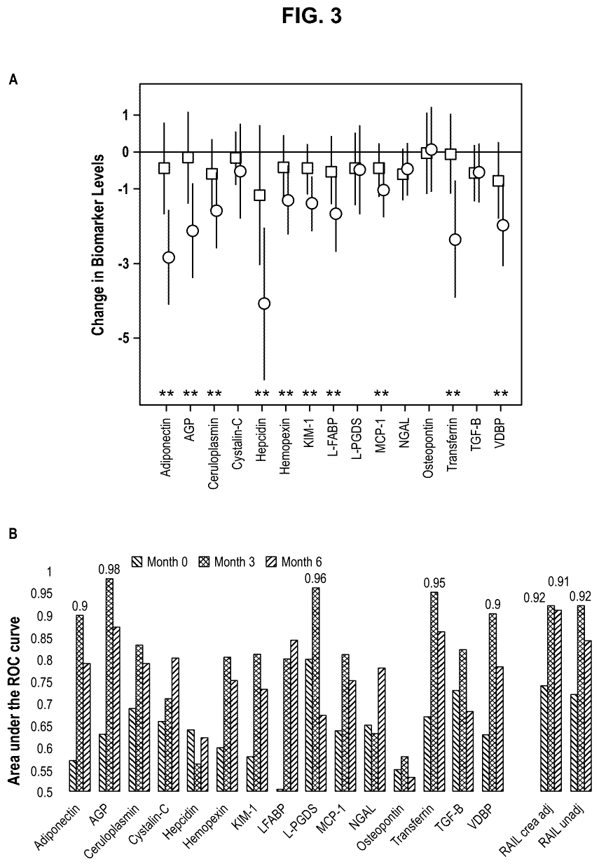

While FIG. 2 describes differences in the mean levels of the biomarkers between responders and non-responders, FIG. 3, Panel (A) depicts changes of biomarker concentrations from baseline to month 3.

With the exception of osteopontin, absolute levels of all of the biomarkers decreased in both responders and non-responders. The reduction by month 3 was more pronounced among responders. Indeed, adiponectin, AGP, transferrin and VDBP levels declined among responders by >2 logs by month 3 of LN therapy. Irrespective of responder status, hepcidin showed the most profound drop in urine levels, but also the most variability (large standard errors). Interestingly, changes of TGF-β from baseline to month 3 did not significantly differ with responder status.

Accuracy of the Biomarkers to Discriminate Responders from Non-Responders

At the time of biopsy none of the biomarkers nor the RAIL-biomarkers in combination achieved excellent accuracy (AUC>0.8) for anticipating the responder status. As shown in FIG. 3, Panel (B), starting at month 3, adiponectin, AGP, LPGDS, transferrin, VDBP individually also had outstanding ability (AUC>0.9) to anticipate treatment response as early as month 3. The RAIL biomarkers also showed outstanding overall accuracy at month 3 (AUC=0.92) and also at month 6 (AUC=0.91).

### Dependency Of Urine Biomarker Levels on Patient Age, Race, Gender and Extra-Renal Disease Activity

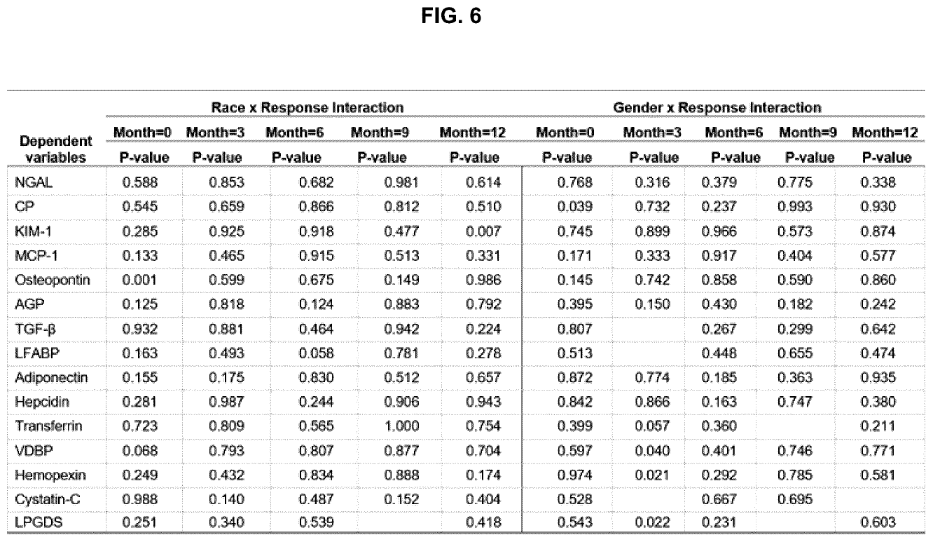

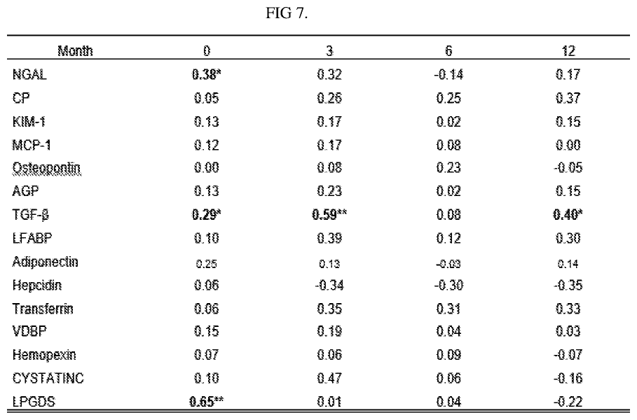

Applicant found that the mean levels of the biomarkers were not importantly or systematically influenced by patient age, race, gender and extra-renal disease activity as measured by the extra-renal SLEDAI (see FIGS. 6 and 7).

### Relevance Of Medication Use on Urine Biomarkers

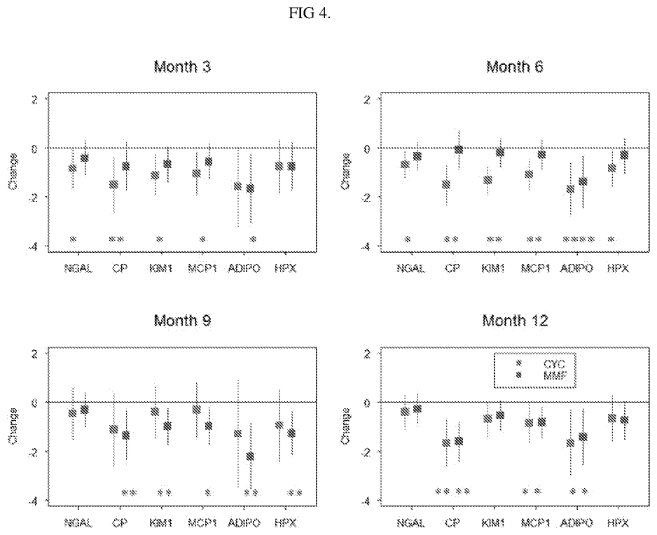

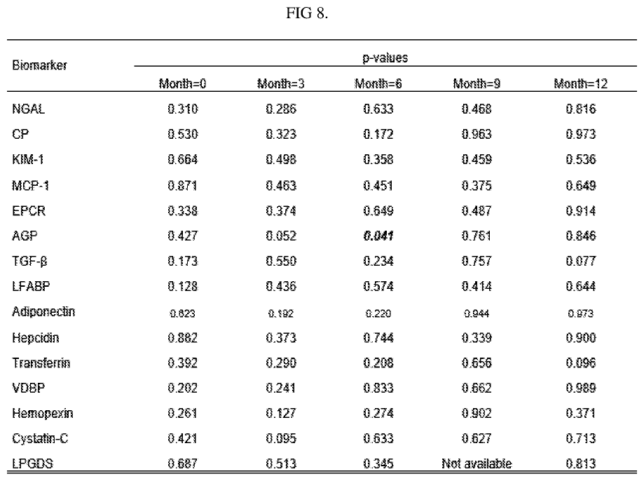

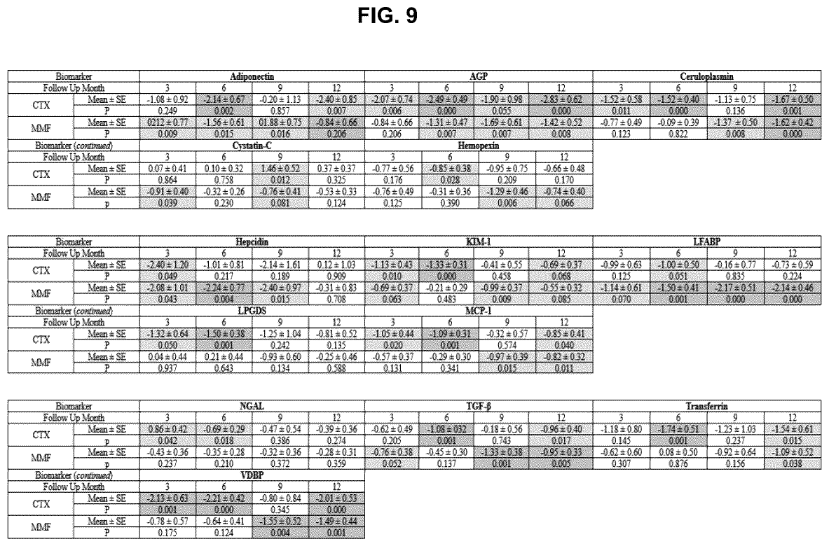

The use of angiotensinogen system blocking medications did not importantly influence biomarker levels (see FIG. 8). Among 47 patients treated with MMF after kidney biopsy, 17 (46%) patients were classified as responders. Of the 33 patients initially treated with intravenous CTX, 15 (40.5%) patients responded to therapy. Early decline of the RAIL-biomarker levels occurred more rapidly among patients treated with CTX as compared to those receiving MMF (FIG. 4). The same held true for most of other biomarkers considered in this study (FIG. 9).

### Discussion

Currently, accurate assessment of LN activity requires a kidney biopsy, and response to LN therapy in children is generally assessed without confirmation by repeat kidney biopsy. When considering a pool of highly promising biomarkers, Applicant confirmed that select biomarkers which reflect LN histological activity, i.e. those included in the RAIL, are suited to predict response to LN therapy. Applicant found transferrin, AGP, and TGF-β levels to be early indicators of LN response to therapy, while LPGDS seemed especially useful to capture improvement of pure membranous LN.

Achieving complete or even partial response to LN therapy often requires more than 6 months. However, both the ACR and The European League against Rheumatism recommend adjusting a chosen LN therapy for questionable LN improvement at 3 months (30, 31). Applicant confirmed that several of the biomarkers previously described by our group and others can serve as “early biomarkers” which help anticipate patients who are at high risk for non-response (10-16).

Among the most promising of these early biomarkers when measured at the time of kidney biopsy are TGF-β, transferrin, ceruloplasmin and AGP. However, different from the other biomarkers, decline of TGF-β levels over time are similar among responders and non-responders. This may support the notion that TGF-β is a risk factor of LN damage. Indeed, it has been recognized that LN chronicity progresses even in patients who respond to LN therapy (32) and that TGF-β promotes scarring of injured glomeruli and tubulointerstitium through accelerated matrix deposition (33).

Applicant previously identified high levels of LFABP, MCP-1 and transferrin at the time of kidney biopsy as risk factors of kidney damage (10). The findings of this study are in line with these earlier observations that high urine levels of MCP-1 and transferrin are risk factors of non-response to LN therapy, with continued renal inflammation increasing the risk of LN damage.

Earlier studies reported that LFABP is a sensitive indicator of acute and chronic tubulointerstitial injury and that increasing urine levels of LFABP are associated with declining renal function (34). Our results suggest that neither baseline levels of LFABP nor absolute changes of LFABP from baseline to month 3 are useful for differentiating between responders and non-responders. Further studies are needed to evaluate its role as a marker of LN damage and interstitial injury with LN.

Several studies showed that LPGDS is strongly associated with GFR decline and ESRD (35, 36). LPGDS is likely locally produced by cells of the proximal tubules, loop of Henle, and glomerulus (37). To the best of our knowledge an association with membranous LN course has not been reported and will need further investigation. Our earlier research suggested that LN biomarkers reflect the diverse histopathological changes observed with LN (10, 16). Therefore, it is not surprising that changes in LN biomarkers with therapy would be influenced by LN severity.

Using a limited number of urine biomarkers that relate to LN histology and can predict LN flares has been the underlying principle for the selection of the RAIL biomarkers (adiponectin, ceruloplasmin, hemopexin, KIM-1, MCP-1, NGAL) (10, 11, 16). Based on the findings of the current study, consideration of also AGP, LPGDS, and transferrin seem sensible for comprehensively capturing LN response over time.

In the past it has been customary to standardize the urine concentrations of biomarkers using urine creatinine or total urine protein. The former is problematic especially in pediatrics where muscle mass is markedly increasing during adolescence, particularly among males; the latter has not been proven useful in our hands either (9). In line with our previous research, Applicant confirm that creatinine adjustment of the levels of urine biomarkers is not necessary to accurately capture LN activity (9, 28).

MMF and cyclophosphamide are commonly considered equivalent in their effectiveness for treating LN in adults (38). Applicant observed several urine biomarkers to decrease slower with MMF as compared to CTX therapy in the children with LN. This might suggest that MMF was dosed inappropriately in children with LN, given that thorough dose-finding studies for proper MMF use in pediatric LN therapy are lacking and marked variability in the active metabolites of MMF are well described in the literature with traditional body-surface based dosing (39).

Angiotensinogen system blocking medications are recommended for patients with LN and marked proteinuria (22, 30, 31). Although use of such medications will alter the PC-ratio, currently the leading laboratory measure used to gauge response to LN therapy, this is not the case for the biomarkers included in this study. Likewise, Applicant did not find any important racial differences in the biomarker levels or a consistent association with extra-renal SLE activity. Taken together these findings are in line with our previous research (10, 11, 16) and confirm the robustness of the proposed LN biomarkers to reflect LN activity over time.

A special strength of our study is that Applicant included children and young adults who lack common age-related kidney pathology which has the potential to influence biomarker identification and verification. Nonetheless, the usefulness of any biomarker found in a pediatric cohort needs to be robust enough to still be useful in adult patient populations. The latter has been shown for the RAIL biomarkers in the past albeit in smaller studies as well as for transferrin, ceruloplasmin and LPGDS (9, 36, 40, 41). Applicant consider the prospective collection of the study cohort with strictly controlled procedures to sample and store urine samples and their testing in a CLIA certified laboratory another strength of our study.

Limitations of our studies include the relatively small sample size, although our cohort constitutes one the largest cohorts to prospectively undergo biomarker evaluation. Small sample sizes are difficult to avoid in pediatric studies in general and pediatric orphan diseases, such as LN in children, in particular.

In summary, Applicant identified a limited number of urine biomarkers that are suited to anticipate response of LN to therapy. If confirmed in large independent cohorts, these “early” biomarkers may prove invaluable for the identification of patients at risk of poor LN outcomes due to their relative resistance to standard therapies and assist in personalizing and optimizing LN care.

### REFERENCES

1. Faurschou M, Starklint H, Halberg P, Jacobsen S. Prognostic factors in lupus nephritis: diagnostic and therapeutic delay increases the risk of terminal renal failure. J Rheumatol. 2006;33 (8): 1563-9.
2. Hugle B, Silverman E D, Tyrrell P N, Harvey E A, Hébert D, Benseler S M. Presentation and outcome of paediatric membranous non-proliferative lupus nephritis. Pediatric Nephrology. 2015;30 (1): 113-21.
3. Rianthavorn P, Buddhasri A. Long-term renal outcomes of childhood-onset global and segmental diffuse proliferative lupus nephritis. Pediatric Nephrology. 2015;30 (11): 1969-76.
4. Ilori T O, Enofe N, Oommen A, Cobb J, Navarrete J, Adedinsewo D A, et al. Comparison of Outcomes between Individuals with Pure and Mixed Lupus Nephritis: A Retrospective Study. PloS one. 2016;11 (6):e0157485.
5. Ruggiero B, Vivarelli M, Gianviti A, Benetti E, Peruzzi L, Barbano G, et al. Lupus nephritis in children and adolescents: results of the Italian Collaborative Study. Nephrol Dial Transplant. 2013;28(6):1487-96.
6. Hiraki L T, Lu B, Alexander S R, Shaykevich T, Alarcon G S, Solomon D H, et al. End-stage renal disease due to lupus nephritis among children in the US, 1995-2006. Arthritis Rheum. 2011;63(7):1988-97.
7. Bennett M, Brunner H I. Biomarkers and updates on pediatrics lupus nephritis. Rheum Dis Clin North Am. 2013;39(4):833-53.
8. Mina R, Abulaban K, Klein-Gitelman M S, Eberhard B A, Ardoin S P, Singer N, et al. Validation of the Lupus Nephritis Clinical Indices in Childhood-Onset Systemic Lupus Erythematosus. Arthritis Care Res (Hoboken). 2016;68(2):195-202.
9. Gulati G, Abulaban K, Ying J, Song H, Zhang X, Ma Q, et al. Urinary Biomarker Based” a-RAIL” (sic) Study in Adults with Lupus Nephritis. ARTHRITIS & RHEUMATOLOGY; 2015: WILEY-BLACKWELL 111 RIVER ST, HOBOKEN 07030-5774, NJ USA; 2015.
10. Abulaban K M, Song H, Zhang X, Kimmel P L, Kusek J W, Nelson R G, et al. Predicting decline of kidney function in lupus nephritis using urine biomarkers. Lupus. 2016.
11. Hinze C H, Suzuki M, Klein-Gitelman M, Passo M H, Olson J, Singer N G, et al. Neutrophil gelatinase-associated lipocalin is a predictor of the course of global and renal childhood-onset systemic lupus erythematosus disease activity. Arthritis Rheum. 2009;60(9):2772-81.
12. Rovin B H, Song H, Birmingham D J, Hebert L A, Yu C Y, Nagaraja H N. Urine chemokines as biomarkers of human systemic lupus erythematosus activity. J Am Soc Nephrol. 2005;16(2):467-73.
13. Brunner H I, Mueller M, Rutherford C, Passo M H, Witte D, Grom A, et al. Urinary neutrophil gelatinase-associated lipocalin as a biomarker of nephritis in childhood-onset systemic lupus erythematosus. Arthritis Rheum. 2006;54(8):2577-84.
14. Suzuki M, Wiers K M, Klein-Gitelman M S, Haines K A, Olson J, Onel K B, et al. Neutrophil gelatinase-associated lipocalin as a biomarker of disease activity in pediatric lupus nephritis. Pediatr Nephrol. 2008;23(3):403-12.
15. Rovin B H. The chemokine network in systemic lupus erythematous nephritis. Front Biosci. 2008; 13:904-22.
16. Brunner H I, Bennett M R, Mina R, Suzuki M, Petri M, Kiani A N, et al. Association of noninvasively measured renal protein biomarkers with histologic features of lupus nephritis. Arthritis Rheum. 2012;64(8):2687-97.
17. Silva C A, Avcin T, Brunner H I. Taxonomy for systemic lupus erythematosus with onset before adulthood. Arthritis Care Res (Hoboken). 2012;64(12): 1787-93.
18. Schwartz G J, Haycock G B, Edelmann C M, Jr., Spitzer A. A simple estimate of glomerular filtration rate in children derived from body length and plasma creatinine. Pediatrics. 1976;58(2):259-63.
19. Kasitanon N, Fine D M, Haas M, Magder L S, Petri M. Estimating renal function in lupus nephritis: comparison of the Modification of Diet in Renal Disease and Cockcroft Gault equations. Lupus. 2007;16(11):887-95.
20. Ibanez D, Gladman D D, Urowitz M B. Adjusted mean Systemic Lupus Erythematosus Disease Activity Index-2K is a predictor of outcome in SLE. J Rheumatol. 2005;32(5):824-7.
21. Yee C S, Farewell V, Isenberg D A, Rahman A, Teh L S, Griffiths B, et al. British Isles Lupus Assessment Group 2004 index is valid for assessment of disease activity in systemic lupus erythematosus. Arthritis Rheum. 2007;56(12):4113-9.
22. Mina R, von Scheven E, Ardoin S P, Eberhard B A, Punaro M, Ilowite N, et al. Consensus treatment plans for induction therapy of newly diagnosed proliferative lupus nephritis in juvenile systemic lupus erythematosus. Arthritis Care Res (Hoboken). 2012;64(3):375-83.
23. Weening J J, D′Agati V D, Schwartz M M, Seshan S V, Alpers C E, Appel G B, et al. The classification of glomerulonephritis in systemic lupus erythematosus revisited. Kidney international. 2004;65 (2):521-30.
24. Weening J J, D′Agati V D, Schwartz M M, Seshan S V, Alpers C E, Appel G B, et al. The classification of glomerulonephritis in systemic lupus erythematosus revisited. J Am Soc Nephrol. 2004;15(2):241-50.
25. Renal Disease Subcommittee of the American College of Rheumatology Ad Hoc Committee on Systemic Lupus Erythematosus Response C. The American College of Rheumatology response criteria for proliferative and membranous renal disease in systemic lupus erythematosus clinical trials. Arthritis Rheum. 2006;54(2):421-32.
26. Schwartz G J, Work D F. Measurement and estimation of GFR in children and adolescents. Clinical Journal of the American Society of Nephrology. 2009;4(11):1832-43.
27. Chaturvedi S, Farmer T, Kapke G F. Assay validation for KIM-1: human urinary renal dysfunction biomarker. Int J Biol Sci. 2009;5(2):128-34.
28. Brunner H I, Bennett M R, Abulaban K, Klein-Gitelman M S, O′Neil K M, Tucker L, et al. Development of a Novel Renal Activity Index of Lupus Nephritis in Children and Young Adults. Arthritis Care Res (Hoboken). 2016;68(7):1003-11.
29. Austin H A, 3rd, Muenz L R, Joyce K M, Antonovych T T, Balow J E. Diffuse proliferative lupus nephritis: identification of specific pathologic features affecting renal outcome. Kidney Int. 1984;25(4):689-95.
30. Hahn B H, McMahon M A, Wilkinson A, Wallace W D, Daikh D I, Fitzgerald J D, et al. American College of Rheumatology guidelines for screening, treatment, and management of lupus nephritis. Arthritis care & research. 2012;64(6):797-808.
31. Bertsias G K, Tektonidou M, Amoura Z, Aringer M, Bajema I, Berden J H, et al. Joint European League Against Rheumatism and European Renal Association-European Dialysis and Transplant Association (EULAR/ERA-EDTA) recommendations for the management of adult and paediatric lupus nephritis. Annals of the rheumatic diseases. 2012;71(11):1771-82.
32. Davidson A. What is damaging the kidney in lupus nephritis? Nat Rev Rheumatol. 2016;12(3):143-53.
33. Border W A. Transforming growth factor-beta and the pathogenesis of glomerular diseases. Curr Opin Nephrol Hypertens. 1994;3(1):54-8.
34. Tanaka T, Doi K, Maeda-Mamiya R, Negishi K, Portilla D, Sugaya T, et al. Urinary L-type fatty acid-binding protein can reflect renal tubulointerstitial injury. Am J Pathol. 2009;174(4):1203-11.
35. White C A, Ghazan-Shahi S, Adams M A. β-Trace protein: a marker of GFR and other biological pathways. American Journal of Kidney Diseases. 2015;65(1):131-46.
36. Dajak M, Ignjatović S, Stojimirović B, Gajić S, Majkić-Singh N. Beta-trace protein as a marker of renal dysfunction in patients with chronic kidney disease: comparison with other renal markers. Journal of Medical Biochemistry. 2010;29(2):66-72.
37. White C A, Ghazan-Shahi S, Adams M A. beta-Trace protein: a marker of GFR and other biological pathways. Am J Kidney Dis. 2015;65(1):131-46.
38. Henderson L K, Masson P, Craig J C, Roberts M A, Flanc R S, Strippoli G F, et al. Induction and maintenance treatment of proliferative lupus nephritis: a meta-analysis of randomized controlled trials. American Journal of Kidney Diseases. 2013;61(1):74-87.
39. Sagcal-Gironella A C, Fukuda T, Wiers K, Cox S, Nelson S, Dina B, et al. Pharmacokinetics and pharmacodynamics of mycophenolic acid and their relation to response to therapy of childhood-onset systemic lupus erythematosus. Semin Arthritis Rheum. 2011;40(4):307-13.
40. Abulaban K M, Song H, Zhang X, Kimmel P L, Kusek J W, Nelson R G, et al. Predicting decline of kidney function in lupus nephritis using urine biomarkers. Lupus. 2016;25(9):1012-8.
41. Zhang X, Nagaraja H N, Nadasdy T, Song H, Mckinley A, Prosek J, et al. A composite urine biomarker reflects interstitial inflammation in lupus nephritis kidney biopsies. Kidney international. 2012;81(4):401-6.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1-20. (canceled)

21. A non-invasive method for forecasting response to lupus nephritis (LN) therapy in an individual diagnosed with Class V (membranous) LN, comprising:
a. detecting a level of lipocalin-like prostaglandin D synthase (LPGDS) in the individual; and
b. classifying the individual as an LN therapy non-responder or an LN therapy responder based on the level of LPGDS; and

22. The method of claim 21, wherein the level is determined in a urine sample obtained from the individual.

23. The method of claim 22, wherein the sample is obtained at a timepoint selected from time of biopsy (month 0), at about three months after biopsy (month 3), or at about six months after biopsy (month six), or combinations thereof.

24. The method of claim 21, wherein if the individual is an LN therapy non-responder, the individual is treated with an alternative therapy or subjected to a repeat biopsy, and if the individual is an LN therapy responder, an initial LN therapy treatment is maintained.

25. The method of claim 21, further comprising comparing the level of LPGDS to a control value, wherein an increase in the level of LPGDS indicates that the individual is likely to be a non-responder, and wherein a decrease in the level of LPGDS compared to the control value indicates that the individual is likely to be a responder.

26. The method of claim 21, further comprising detecting at least one or both of vitamin D binding protein (VDBP) and adiponectin.

27. The method of claim 26, wherein the urine sample is obtained at three months post-treatment initiation, or six months post-treatment initiation.

28. The method of claim 21, wherein the initial LN therapy treatment further comprises an angiotensin system-blocking drug.

29. The method of claim 21, wherein the LN is proliferative LN.

30. The method of claim 21, wherein the individual is less than 18 years of age.

31. The method of claim 21, wherein at least one step is calculated using a computer.

32. The method of claim 22, wherein the detecting comprises contacting the sample with a composition comprising a detection agent specific for LPGDS and optionally, one or both of a detection agent specific for VDBP and a detection agent specific for adiponectin.

33. The method of claim 32, wherein the detection agent is an antibody.

34. The method of claim 21, further comprising administering an active agent selected from mycophenolate mofetil (MMF), cyclophosphamide (CTX), and combinations thereof, to the LN therapy responder.

35. A kit for forecasting response to lupus nephritis (LN) therapy in individual diagnosed with class V LN, comprising a set of detection agents capable of detecting an expression product of LPGDS, and optionally, VDBP, and adiponectin.

36. A composition comprising a plurality of detection agents, the detection agents comprising a detection agent specific for LPGDS, and, optionally, one or both of a detection agent specific for VDBP and a detection agent specific for adiponectin.

37. The composition of claim 36, wherein the detection agent is provided in a form selected from a solution-based composition or a substrate-based composition.
